(19) United States
(12) Plant Patent Application Publication
(10) Pub. No.: US 2002/0073464 P1
(43) Pub. Date: Jun. 13, 2002

(54) VARIETY OF GERANIUM PLANT NAMED 'PACTWO'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/739,022

(22) Filed: Dec. 8, 2000

(51) Int. Cl. A01H 5/00

(52) U.S. Cl. PLT/329

(57) ABSTRACT

A new and distinct pelargonium plant with exciting red-purple colored flowers above medium green foliage. The plant flowers are suitable for pot planting and as a bedding plant.

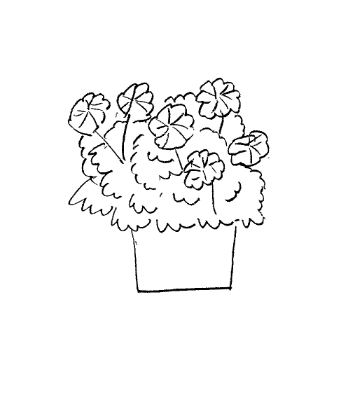

### BACKGROUND OF THE INVENTION

The present invention comprises a new and distinct cultivar of Pelargonium×hortorum known by the varietal name ‘Pactwo’. The new variety was discovered in 2000 in a selective breeding program in Dresden, Germany. The new variety is a mutation of ‘Penbalu’ (PEL 1416). The new variety exhibits the growth and flower color of its parent, but differs in its earlier flowering and improved resistance to heat. The new variety was first asexually reproduced by cuttings in Dresden, Germany in 2000. The new variety has been trial and field tested at Connellsville and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to the R.H.S. Colour Chart of The Royal Horticultural Society of London.

Plant:

Classification:

Botanical.—Pelargoniump×hortorum.

Commercial.—Zonal geranium.

Form.—Erect.

Height from media surface to top of foliage.—35-40 cm.

Height from media surface to top of umbel.—50 cm.

Growth.—Good branching.

Strength.—Does not require artificial support.

Response time.—3.5 weeks.

Leaves:

Size.—8-10 cm long.

Shape.—Cordate.

Margin.—Emarginate.

Texture.—Velvety.

Color.—Green Group 137A with prominent zone.

Ribs and veins:

Color.—Brighter than leaf surface.

Petioles:

Color.—Medium green.

Length.—4-6 cm.

Stem/branches:

Color.—Medium Green.

Internode length.—3-4 cm.

The bud:

Shape when just showing color:

Overall.—Spherical.

Individual bud.—Spherical.

Color.—Yellow-Green Group 144A.

Size when just showing color:

Umbel.—10-12 cm across.

Individual bud.—1.3-1.5 cm long; 1.0 cm wide.

Inflorescence:

Blooming habit.—Semi-double.

Size of fully open bloom.—4.5 cm across.

Borne.—Flowers on umbel; umbel on pedicel; pedicel on peduncle.

Flowers:

Form.—Zygomorph.

Number of petals.—7-9.

Size.—2.0-2.5 cm across.

Petals:

Color:

Upper surface.—Red-Purple Group 57A.

Lower surface.—Red-Purple Group 57A.

Texture and appearance.—Velvety.

Petaloids:

Quantity.—3.

Shape.—Flakes.

Color:

Upper surface.—Red-Purple Group 57A.

Lower surface.—Red-Purple Group 57A.

Pedicel:

Length.—2.5-3.0 cm.

Color.—Reddish.

Peduncle:

Length.—9-11.0 cm.

Color.—Green-reddish.

Disease/pest resistance.—No unusual susceptibility to diseases or pests has been noted to date.

Lasting Quality.—Long-lasting; 4 weeks per umbel.

Reproductive organs:

Stamens:

Anthers.—3 mm long.

Filaments:

Length.—0.5 cm maximum.

Color.—Pink.

Pollen.—Yellow.

Pistils:

Number.—Five.

Length.—0.3-0.4 cm.

Stigma.—Red.

Style.—0.8 cm long.

Ovaries:

Color.—Greenish.

Length.—0.4 cm.

Fruit.—Rarely present.

### GENERAL CHARACTERISTICS

1. Suitable for pot and as bedding plants.

2. Prominent zoning present.

3. Red-purple flower color.

4. Heat resistant.

I claim:

1. A new and distinct variety of geranium plant substantially as shown and described.
